(19) United States
(12) Patent Application Publication
(10) Pub. No.: US 2010/0092550 A1
(43) Pub. Date: Apr. 15, 2010

(54) ALKA-EEZE

(76) Inventor: NELSON CABRAL, Port Moody (CA)

Correspondence Address: James Ray & Associates, 2640 Pitcairn Road, Monroeville, PA, 15146, US

(21) Appl. No.: 12/403,631

(22) Filed: Mar. 13, 2009

Related U.S. Application Data
(60) Provisional application No. 61/196,010, filed on Oct. 14, 2008.

(51) Int. Cl. A61K 9/52; A61K 9/22; A61K 33/10; A61P 1/04

(52) U.S. Cl. 424/457; 424/468; 424/717

(57) ABSTRACT

A composition for providing relief from acidity. The composition comprises sodium bicarbonate for use in neutralizing acids. There is one of a capsule and a tablet for enclosing the sodium bicarbonate therein, such one of such capsule and such tablet being a delayed release and a timed release system so as to delay the dissolution of the sodium bicarbonate until the sodium bicarbonate is in one of a small intestine and a large intestine.

### CROSS REFERENCE TO RELATED APPLICATIONS

This application is closely related to and claims benefit from U.S. Provisional Application Ser. No. 61/196,010 filed Oct. 14, 2008.

### FIELD OF THE INVENTION

The present invention relates, in general, to a antacid, and, more particularly, the present invention relates to a time release capsule/tablet containing sodium bicarbonate which does not dissolve in the stomach but is delayed such that the capsule/tablet will dissolve in one of the small and/or large intestine thereby bypassing the acidic environment of the stomach.

### BACKGROUND OF THE INVENTION

Sodium bicarbonate is an extremely powerful alkaline and is humans taking the compound directly will reduce their stomach's acid, which can interfere with the common digestive process normally occurring in the small and large intestines. Presently, the common method of ingesting sodium bicarbonate forces people to dissolve the compound in water which they drink or is taken as a tablet with a glass of water. Since the sodium bicarbonate is mostly consumed by the stomach's acid through this method, less of the compound is left available to affect the remainder of the body.

Thus, it would be advantageous if there were a bicarbonate compound that could bypass the stomach and dissolve in either the small or large intestine.

### SUMMARY OF THE INVENTION

In a first aspect the present invention provides a composition for providing relief from acidity. The composition comprises sodium bicarbonate for use in neutralizing acids. One of a capsule and a tablet is used for enclosing the sodium bicarbonate therein, such one of such capsule and such tablet being a delayed release and a timed release system so as to delay the dissolution of the sodium bicarbonate until the sodium bicarbonate is in one of the small intestine and the large intestine.

In a second aspect, the present invention provides a composition taken as a suppository for providing relief from acidity. The composition comprises sodium bicarbonate for use in neutralizing acids. One of a capsule and a tablet is used for enclosing the sodium bicarbonate therein, such one of such capsule and such tablet being a timed release system so as to prevent excessive rapid dissolution of such sodium bicarbonate in the large intestine.

### OBJECTS OF THE INVENTION

It is, therefore, one of the primary objects of the present invention to provide a timed release tablet/capsule containing sodium bicarbonate that dissolves in the intestines.

Another object of the present invention is to provide a timed release tablet/capsule that bypasses the stomach and does not dissolve until it reaches the small intestines.

Still another object of the present invention is to provide a timed release tablet/capsule that begins to dissolve in the small intestines and continues dissolving in the large intestines.

Yet another object of the present invention is to provide a timed release tablet/capsule that can be used as a suppository.

In addition to the various objects and advantages of the invention which have been described in some specific detail above it should be noted that various other objects and advantages of the present invention will become more readily apparent to those persons who are skilled in the relevant art from the following more detailed description, particularly when such description is taken in conjunction with the appended claims.

### BRIEF DESCRIPTION OF THE DRAWINGS

FIG. 1 is a perspective view of a persons intestines showing the tablet or capsule in the small intestines.

FIG. 2 is a perspective view of a persons intestines showing the capsule or tablet in the large intestines.

FIG. 3 is a perspective view of a persons intestines showing a portion of the capsule or tablet being passed through the users rectum.

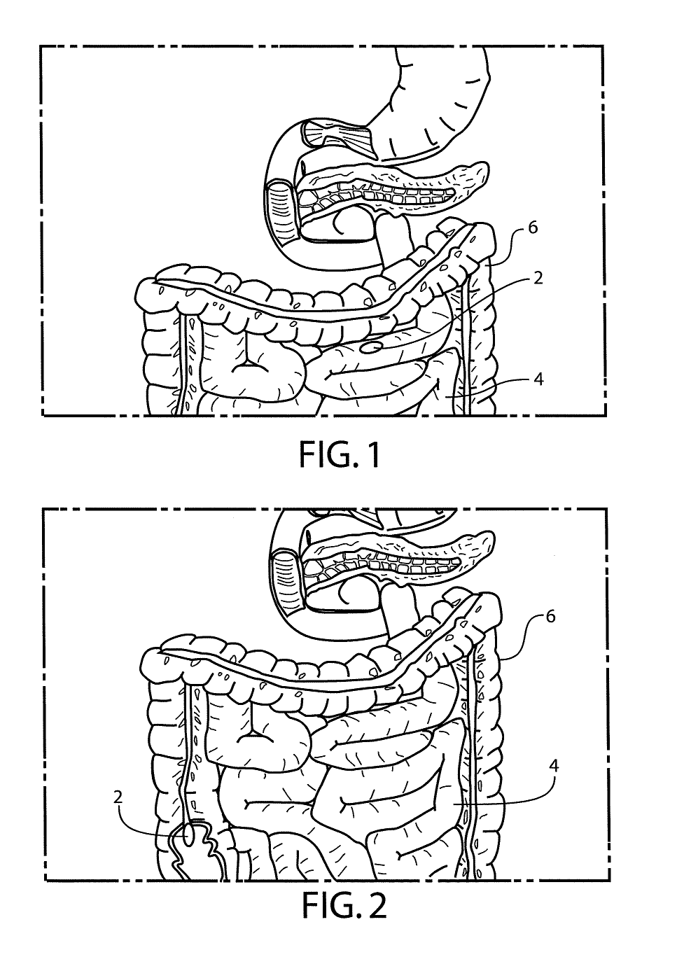

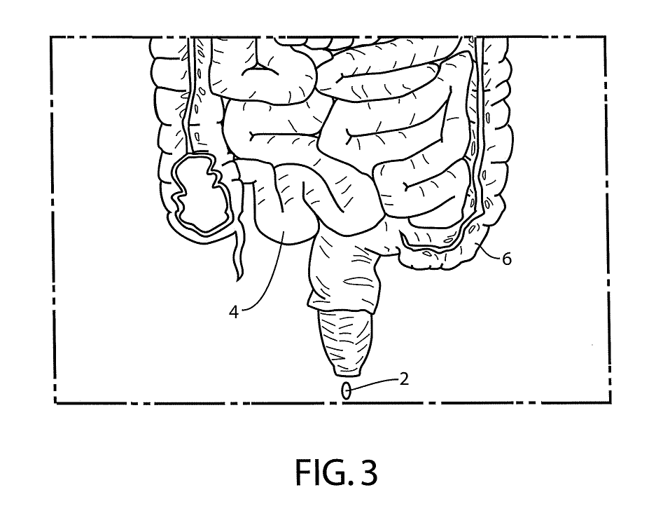

### BRIEF DESCRIPTION OF THE PRESENTLY PREFERRED AND VARIOUS ALTERNATIVE EMBODIMENTS OF THE INVENTION

Prior to proceeding with the more detailed description of the present invention it should be noted that, for the sake of clarity, identical components which have identical functions have been designated by identical reference numerals throughout the several views illustrated in the drawings.

In a first aspect the present invention provides a composition for providing relief from acidity. The composition comprises sodium bicarbonate for use in neutralizing acids. One of a capsule and a tablet 2 for enclosing the sodium bicarbonate therein, such one of such capsule 2 and such tablet 2 being a delayed release and a timed release system so as to delay the dissolution of the sodium bicarbonate until the sodium bicarbonate is in one of the small intestine 4 and the large intestine 6.

In a second aspect, the present invention provides a composition taken as a suppository for providing relief from acidity. The composition comprises sodium bicarbonate for use in neutralizing acids. One of a capsule 2 and a tablet 2 for enclosing the sodium bicarbonate therein, such one of such capsule 2 and such tablet 2 being a timed release system so as to prevent excessive rapid dissolution of such sodium bicarbonate in the large intestine 6.

Medicines are drugs that serve some beneficial purpose in the body by interacting with specific molecules in the body. A medicine will only work if enough of the drug makes it to the target without getting chemically changed or destroyed in the process. Since the body's digestive system can have a great effect on the drug during its journey to the bloodstream, it is important to choose an appropriate system of delivery to make sure there is enough of the active drug available once it reaches the target.

Delayed release and time release are more broadly referred to as controlled release medicines and constitute a separate category of drug delivery systems. There has been much research in using the controlled release system of drug delivery. Until the development of controlled release drug delivery systems, the active drug in a medication was released all at once. This is called the “burst effect” and may not always be the most effective method for the drug to carry out its work in the body. With the advancements made so far in controlled release drug delivery, it is possible to control the rate, time, and even frequency of drug release inside the body. This kind of control can result in longer periods of drug activity in the body, lower frequency of necessary doses, and even the possibility of reduced side effects.

One method of controlling the release of medications results in embedding the active drug in a polymer matrix. This type of release was once controlled only by diffusion, but current research is focused on biodegradable polymer systems that will leave only the medication behind once the polymers have broken down. Scientists have learned that polymers that are hydrophilic, amorphous, have a low-molecular weight, and contain heteroatoms in the backbone are fast to degrade. Each of these factors can be manipulated to adjust the rate of degradation.

In addition to the polymer matrices, block copolymers (a network formed from the joint polymerization of several different monomers) may also be used to form the polymer matrix. The inclusion of multiple polymer segments allows for much variation in mesh size and rate of degradation. Both of these variables can have an effect on the rate of release of a drug from the matrix.

Thus, the present invention provides sodium bicarbonate as an antacid. In the present invention the sodium bicarbonate is disposed in a capsule or a tablet. The capsule or tablet is ingested like a normal pill but it is a delayed release and a timed release system which allows the sodium bicarbonate to dissolve through the capsule/tablet on a time release system. Thus, the sodium bicarbonate is delivered or released in the intestines rather than the stomach and counteracts common over-acidic conditions accompanying the consumption of highly acidic foods such as alcohol, coffee, sugar, and foods containing tomato sauce such as pizza and spaghetti.

Sodium bicarbonate is a very effective lactic acid neutralizer. It also can combat intestinal fungus and the amount of fungus affected by the sodium bicarbonate is directly proportional to the amount of the compound that contacts the fungus. The delayed release portion of the composition prevents the capsule or tablet from dissolving in the stomach. The timed release portion of the capsule or tablet disperses the bicarbonate in the intestines in three different ways. First, it bypasses the stomach and dissolves in the small intestines. Second, it can bypass through the stomach to begin dissolving in the small intestine and finish dissolving in the large intestine. Third, the capsule/tablet can bypass through the stomach, start dissolving in the small and large intestine and then an extremely small portion of the capsule or tablet that did not dissolve can pass through the consumer's rectum.

Without delayed release the capsule/tablet may also be taken as a suppository—via insertion in the rectum—to provide immediate dispersion of the sodium bicarbonate. However, the suppository must be designed with a specific timed release so as to not deposit excess sodium bicarbonate into the intestines too quickly, which may cause irritation to the intestinal lining. Regardless how the capsule/tablet is ingested, it helps reduce acidity in the body by slowly dispersing the sodium bicarbonate in the intestines.

While a presently preferred embodiment and alternate embodiments of the present invention have been described in detail above, it should be understood that various other adaptations and/or modifications of the invention can be made by those persons who are particularly skilled in the art without departing from either the spirit of the invention or the scope of the appended claims.

I claim:

1. A composition for providing relief from acidity, said composition comprising:
(a) sodium bicarbonate for use in neutralizing acids;
(b) one of a capsule and a tablet having each of a predetermined size and a predetermined shape for enclosing said sodium bicarbonate therein, said one of said capsule and said tablet being a delayed release and a timed release system so as to delay the dissolution of said sodium bicarbonate until said sodium bicarbonate is in one of a small intestine and a large intestine.

2. The composition, according to claim 1, wherein said delayed release portion of said one of a capsule and a tablet delays dissolution of said sodium bicarbonate until said one of a capsule and a tablet has passed through a stomach and is in such intestines.

3. The composition, according to claim 1, wherein said timed release portion is designed to control release of said sodium bicarbonate so as to prevent rapid excessive sodium bicarbonate dissolution in said small intestine so as to prevent irritation of intestinal lining.

4. The composition, according to claim 1, wherein said sodium bicarbonate is further used to combat intestinal fungus.

5. The composition, according to claim 1, wherein said predetermined shape of said capsule is substantially ovate.

6. A composition taken as a suppository for providing relief from acidity, said composition comprising:
(a) sodium bicarbonate for use in neutralizing acids;
(b) one of a capsule and a tablet having a predetermined size and a predetermined shape for enclosing said sodium bicarbonate therein, said one of said capsule and said tablet being a timed release system so as to delay excessive rapid dissolution of said sodium bicarbonate in the large intestine.

7. The composition, according to claim 6, wherein said predetermined shape of said capsule is substantially ovate.
